(19) United States
(12) Patent Application Publication
(10) Pub. No.: US 2011/0052509 A1
(43) Pub. Date: Mar. 3, 2011

(54) COMPOSITIONS COMPRISING BASIC AMINO ACID AND SOLUBLE CARBONATE SALT

(75) Inventors: Ravi Subramanyam, Belle Mead, NJ (US); Rajnish Kohli, Hillsborough, NJ (US); Eric A. Simon, Somerset, NJ (US); Richard Scott Robinson, Belle Mead, NJ (US); Richard J. Sullivan, Atlantic Highlands, NJ (US); Donghui Wu, Bridgewater, NJ (US)

(73) Assignee: Colgate-Palmolive Company, New York, NY (US)

(21) Appl. No.: 12/866,644

(22) PCT Filed: Feb. 6, 2009
(86) PCT No.: PCT/US09/33308

§ 371 Date: Nov. 15, 2010

Related U.S. Application Data
(60) Provisional application No. 61/027,424, filed on Feb. 8, 2008.

(51) Int. Cl. A61K 8/21; A61K 8/44; A61Q 11/00

(52) U.S. Cl. 424/52; 424/54

(57) ABSTRACT

This invention relates to compositions comprising a basic amino acid free or salt form and a soluble carbonate or bicarbonate salt.

This application claims the benefit of U.S. Patent Application Ser. No. 61/027,424 filed Feb. 8, 2008, the contents of which are incorporated herein by reference.

### BACKGROUND OF THE INVENTION

Arginine and other basic amino acids have been proposed for use in oral care and are believed to have significant benefits in combating cavity formation and tooth sensitivity. Commercially available arginine-based toothpaste, such as ProClude® or DenClude®, for example, contains arginine bicarbonate; however, such salts are expensive.

Arginine bicarbonate is produced by bubbling carbon dioxide gas through a saturated arginine aqueous solution. However, the efficiency of the existing process needs to be improved. First, the existing process is slow, requiring 24 to 48 hours to complete the reaction. Second, carbon dioxide has very limited solubility in water, and the solution reaches a maximum concentration of about 1.2×10−5 M at room temperature and normal carbon dioxide partial pressure. Second, the solubility of arginine in water is only about 15% weight/weight at room temperature. Producing a concentrated arginine bicarbonate solution (e.g., at least 40%) requires the addition of arginine to the solution, thereby increasing production time and requires constant monitoring of the reaction.

It is therefore desirable to develop compositions and formulations which take advantage of the benefits of arginine, while reducing costs of the ingredients.

### BRIEF SUMMARY OF THE INVENTION

The invention encompasses oral care compositions and methods of using the same that are effective in inhibiting or reducing the accumulation of plaque, reducing levels of acid producing (cariogenic) bacteria, remineralizing teeth, and inhibiting or reducing gingivitis. The invention also encompasses compositions and methods to clean the oral cavity and provide improved methods of promoting oral health and/or systemic health, including cardiovascular health, e.g., by reducing potential for systemic infection via the oral tissues.

The invention thus comprises Composition 1.0, an oral care composition, e.g., a dentifrice, comprising a basic amino acid, e.g., arginine, in free or salt form, arginine hydrochloride, together with a soluble carbonate salt, e.g., sodium carbonate, sodium bicarbonate or mixtures thereof, wherein a bicarbonate of the basic amino acid is formed in situ.

By “soluble carbonate salt” meant any soluble salt formed by carbonic acid or dissolved carbon dioxide. In aqueous solution, the carbonate ion, bicarbonate ion, carbon dioxide, and carbonic acid form a dynamic equilibrium. The term “carbonate” as used herein thus encompasses bicarbonate (HCO₃⁻) and carbonate (CO₃²⁻) forms and mixtures thereof. Soluble carbonate salts thus include, e.g., potassium carbonate, potassium bicarbonate, sodium carbonate, and sodium bicarbonate.

By “in situ” is meant that the bicarbonate salt of the basic amino acid is formed within the composition.

Composition 1.0 thus includes for example any of the following compositions:

1.0.1. Composition 1.0 wherein the basic amino acid is arginine, lysine, citrullene, ornithine, creatine, histidine, diaminobutanoic acid, diaminoproprionic acid, salts thereof and/or combinations thereof.
1.0.2. Composition 1.0 or 1.0.1 wherein the basic amino acid has the L-configuration.
1.0.3. Any of the preceding compositions wherein the basic amino acid is arginine.
1.0.4. Any of the preceding compositions wherein the basic amino acid is L-arginine.
1.0.5. Any of the preceding compositions wherein the basic amino acid is initially provided partially or wholly in salt form.
1.0.6. Composition 1.0.5 wherein the basic amino acid is in initially provided to the formulation in the form of arginine hydrochloride.
1.0.7. Any of the preceding compositions wherein the soluble carbonate salt is sodium bicarbonate.
1.0.8. Any of the preceding compositions wherein the basic amino acid is present in an amount corresponding to about 0.1—about 20%, e.g., about 1 wt. % to about 10 wt. % of the total composition weight, the weight of the basic amino acid being calculated as free base form.
1.0.9. Composition 1.0.8 wherein the basic amino acid is present in an amount of about 1.5, about 3.75, about 5, or about 7.5 wt. % of the total composition weight.
1.0.10. Any of the preceding compositions comprising a fluoride source, e.g., wherein the fluoride is covalently bound to another atom, e.g., selected from fluorophosphates e.g., sodium monofluorophosphate, fluorosilicates, e.g., sodium fluorosilicate, ammonium fluorosilicate, and fluorosulfates, e.g., hexafluorosulfate, and combinations thereof.
1.0.11. Composition 1.0.10 wherein the fluoride salt is sodium monofluorophosphate.
1.0.12. Any of the preceding compositions wherein a fluoride salt is present in an amount of about 0.01 wt. % to about 2 wt. % of the total composition weight.
1.0.13. Any of the preceding compositions wherein a fluoride salt provides fluoride ion in an amount of about 0.1 to about 0.2 wt. % of the total composition weight.
1.0.14. Any of the preceding compositions wherein a soluble fluoride salt provides fluoride ion in an amount of from about 50 to about 25,000 ppm.
1.0.15. Any of the preceding compositions which is a dentifrice having about 750 to about 2000 ppm available fluoride ion.
1.0.16. Any of the preceding compositions wherein the composition comprises about 1000 to about 1500 ppm fluoride ion.
1.0.17. Any of the preceding compositions wherein the composition comprises about 1450 ppm fluoride ion.
1.0.18. Any of the preceding compositions wherein the pH is about 6 to about 9.
1.0.19. Any of the preceding compositions wherein the pH is about 8 to about 9.
1.0.20. Any of the preceding compositions further comprising an abrasive or particulate.
1.0.21. The immediately preceding composition wherein. the abrasive or particulate is selected from sodium bicarbonate, calcium phosphate (e.g., dicalcium phosphate dihydrate), calcium sulfate, calcium carbonate, hydroxyapatite, precipitated calcium carbonate, silica (e.g., hydrated silica), iron oxide, aluminum oxide, perlite, plastic particles, e.g., polyethylene, and combinations thereof.
1.0.22. The immediately preceding composition wherein the abrasive or particulate is selected from precipitated calcium carbonate, silica (e.g., hydrated silica), and combinations thereof.
1.0.23. Any of the preceding compositions comprising an abrasive in an amount of about 15 wt. % to about 70 wt. % of the total composition weight.
1.0.24. Any of the preceding compositions comprising a small particle abrasive fraction of at least about 5% having a d50 of about less than about 5 micrometers.
1.0.25. Any of the preceding compositions have a RDA of less than about 150, e.g., about 40 to about 140.
1.0.26. Any of the preceding compositions comprising an anionic surfactant.
1.0.27. Any of the preceding compositions wherein the anionic surfactant is selected from sodium lauryl sulfate, sodium ether lauryl sulfate, and mixtures thereof.
1.0.28. Any of the preceding compositions wherein the anionic surfactant is present in an amount of about 0.3% to about 4.5% by weight.
1.0.29. Any of the preceding compositions comprising surfactants selected from anionic, cationic, zwitterionic, and nonionic surfactants, and mixtures thereof.
1.0.30. Any of the preceding compositions comprising at least one humectant.
1.0.31. Any of the preceding compositions comprising at least one humectant, e.g., a polyol, e.g., selected from glycerin, sugar alcohols, (e.g., sorbitol, xylitol), and combinations thereof.
1.0.32. Any of the preceding compositions comprising xylitol.
1.0.33. Any of the preceding compositions comprising at least one polymer.
1.0.34. Any of the preceding compositions comprising at least one polymer selected from polyethylene glycols, polyvinylmethyl ether maleic acid copolymers, polysaccharides (e.g., cellulose derivatives, for example carboxymethyl cellulose, or polysaccharide gums, for example xanthan gum or carrageenan gum), and combinations thereof.
1.0.35. Any of the preceding compositions comprising gum strips or fragments.
1.0.36. Any of the preceding compositions comprising flavoring, fragrance and/or coloring.
1.0.37. Any of the preceding compositions comprising water.
1.0.38. Any of the preceding compositions comprising an antibacterial agent selected from halogenated diphenyl ether (e.g. triclosan), herbal extracts and essential oils (e.g., rosemary extract, tea extract, magnolia extract, thymol, menthol, eucalyptol, geraniol, carvacrol, citral, hinokitol, catechol, methyl salicylate, epigallocatechin gallate, epigallocatechin, gallic acid, miswak extract, sea-buckthorn extract), bisguanide antiseptics (e.g., chlorhexidine, alexidine or octenidine), quaternary ammonium compounds (e.g., cetylpyridinium chloride (CPC), benzalkonium chloride, tetradecylpyridinium chloride (TPC), N-tetradecyl-4-ethylpyridinium chloride (TDEPC)), phenolic antiseptics, hexetidine, octenidine, sanguinarine, povidone iodine, delmopinol, salifluor, metal ions (e.g., zinc salts, for example, zinc citrate, stannous salts, copper salts, iron salts), sanguinarine, propolis and oxygenating agents (e.g., hydrogen peroxide, buffered sodium peroxyborate or peroxycarbonate), phthalic acid and its salts, monoperthalic acid and its salts and esters, ascorbyl stearate, oleoyl sarcosine, alkyl sulfate, dioctyl sulfosuccinate, salicylanilide, domiphen bromide, delmopinol, octapinol and other piperidino derivatives, nicin preparations, chlorite salts; and mixtures of any of the foregoing.
1.0.39. Any of the preceding compositions comprising an anti-inflammatory compound, e.g., an inhibitor of at least one of host pro-inflammatory factors selected from matrix metalloproteinases (MMP's), cyclooxygenases (COX), PGE2, interleukin 1 (IL-1), IL-1β converting enzyme (ICE), transforming growth factor β1 (IGF-β1), inducible nitric oxide synthase (iNOS), hyaluronidase, cathepsins, nuclear factor kappa B (NF-κB), and IL-1 Receptor Associated Kinase (IRAK), e.g, selected from aspirin, ketorolac, flurbiprofen, ibuprofen, naproxen, indomethacin, aspirin, ketoprofen, piroxicam, meclofenamic acid, nordihydoguaiaretic acid, and mixtures thereof.
1.0.40. Any of the preceding compositions comprising an antioxidant, e.g., selected from the group consisting of Co-enzyme Q10, PQQ, Vitamin C, Vitamin E, Vitamin A, anethole-dithiothione, and mixtures thereof.
1.0.41. Any of the preceding compositions comprising triclosan.
1.0.42. Any of the preceding composition comprising triclosan and Zn²⁺ ion source, e.g., zinc citrate.
1.0.43. Any of the preceding compositions comprising triclosan and xylitol.
1.0.44. Any of the preceding compositions comprising triclosan, xylitol, and precipitated calcium carbonate.
1.0.45. Any of the preceding compositions comprising solbrol and chitosan.
1.0.46. Any of the preceding compositions further comprising an anti-calculus agent.
1.0.47. Any of the preceding compositions further comprising an anti-calculus agent which is a polyphosphate, e.g., pyrophosphate, tripolyphosphate, or hexametaphosphate, e.g., in sodium salt form.
1.0.48. Any of the preceding posit comprising an antibacterial agent in an amount of about 0.01 to about 5 wt. % the total composition weight.
1.0.49. Any of the preceding compositions comprising triclosan in an amount of about 0.01 to about 1 wt. percent of the total composition weight.
1.0.50. Any of the preceding compositions comprising triclosan in an amount of about 0.3% of the total composition weight.
1.0.51. Any of the preceding compositions comprising a whitening agent.
1.0.52. Any of the preceding compositions comprising a whitening agent selected from a whitening active selected from the group consisting of peroxides, metal chlorites, perborates, percarbonates, peroxyacids, hypochlorites, and combinations thereof.
1.0.53. Any of the preceding compositions further comprising hydrogen peroxide or a hydrogen peroxide source, e.g., urea peroxide or a peroxide salt or complex (e.g., such as peroxyphosphate, peroxycarbonate, perborate, peroxysilicate, or persulphate salts; for example calcium peroxyphosphate, sodium perborate, sodium carbonate peroxide, sodium peroxyphosphate, and potassium persulfate), or hydrogen peroxide polymer complexes such as hydrogen peroxide-polyvinyl pyrrolidone polymer complexes.
1.0.54. Any of the preceding compositions further comprising an agent that interferes with or prevents bacterial attachment, e.g., solbrol or chitosan.
1.0.55. Any of the preceding compositions further comprising a source of calcium and phosphate selected from (i) calcium-glass complexes, e.g., calcium sodium phosphosilicates, and (ii) calcium-protein complexes, e.g., casein phosphopeptide-amorphous calcium phosphate.
1.0.56. Any of the preceding compositions further comprising a soluble calcium salt, e.g., selected from calcium sulfate, calcium chloride, calcium nitrate, calcium acetate, calcium lactate, and combinations thereof.
1.0.57. Any of the preceding compositions further comprising a physiologically acceptable potassium salt, e.g., potassium nitrate or potassium chloride, in an amount effective to reduce dentinal sensitivity.
1.0.58. Any of the preceding compositions comprising from about 0.1% to about 7.5% of a physiologically acceptable potassium salt, e.g., potassium nitrate and/or potassium chloride.
1.0.59. Any of the preceding composition which is a toothpaste comprising triclosan; an anionic surfactant, and/or a compatible soluble fluoride salt, e.g., sodium monofluorophosphate.
1.0.60. Any of the preceding compositions effective upon application to the oral cavity, e.g., with brushing, to (i) reduce or inhibit formation of dental caries, (ii) reduce, repair or inhibit pre-carious lesions of the enamel, e.g., as detected by quantitative light-induced fluorescence (QLF) or electrical caries measurement (ECM), (iii) reduce or inhibit demineralization and promote remineralization of the teeth, (iv) reduce hypersensitivity of the teeth, (v) reduce or inhibit gingivitis, (vi) promote healing of sores or cuts in the mouth, (vii) reduce levels of acid producing bacteria, (viii) to increase relative levels of arginolytic bacteria, (ix) inhibit microbial biofilm formation in the oral cavity, (x) raise and/or maintain plaque pH at levels of at least pH 5.5 following sugar challenge, (xi) reduce plaque accumulation, (xi) relieve or reduce dry mouth, (xiii) clean the teeth and oral cavity (xiv) reduce erosion, (xv) whiten teeth, (xvi) immunize the teeth against cariogenic bacteria; and/or (xvii) promote systemic health, including cardiovascular health, e.g., by reducing potential for systemic infection via the oral tissues.
1.0.61. A composition obtained or obtainable by combining the ingredients as set forth in any of the preceding compositions.
1.0.62. Any of the preceding compositions in a form selected from mouthrinse, toothpaste, tooth gel, tooth powder, non-abrasive gel, mousse, foam, mouth spray, lozenge, oral tablet, dental implement, and pet care product.
1.0.63. Any of the preceding compositions wherein the composition is toothpaste.
1.0.64. Any of the preceding compositions wherein the composition is a toothpaste optionally further comprising one or more of one or more of water, abrasives, surfactants, foaming agents, vitamins, polymers, enzymes, humectants, thickeners, antimicrobial agents, preservatives, flavorings, colorings and/or combinations thereof.
1.0.65. Any of the preceding compositions 1.0-1.0.61 wherein the composition is a mouthwash.
1.0.66. Any of the preceding compositions further comprising a breath freshener, fragrance or flavoring.
1.0.67. Any of the preceding compositions positions when made by a process of Method 2.0-2.5.

The present invention also encompasses method 2.0, a method for preparing an oral composition comprising mixing a basic ammo acid in free or salt form and a carbonate salt. Optionally the composition can be adjusted to a pH of about 8.5 to about 9.5. Further, secondary materials can be admixed with to the composition to form an oral composition, e.g., according to any of compositions 1.0-1.0.61 above.

Method 2.0 thus includes. e.g., the following embodiments:

2.1 Method 2.0 wherein the carbonate salt is selected from sodium carbonate and sodium bicarbonate.

2.2 Method 2.0 or 2.1 wherein the basic amino acid is selected from arginine, lysine, citrullene, ornithine, creatine, histidine, diaminobutanoic acid, diaminoproprionic acid, in free or salt form, and/or combinations thereof.

2.3 Method 2.2 wherein the basic amino acid is arginine.

2.4 Method 2.3 wherein the arginine is in a form selected from free base, hydroxide, hydrochloride, and mixtures thereof.

2.5 Any of the preceding methods wherein the premix is adjusted to about pH 9.

The invention thus further encompasses methods (Method 3) to (i) reduce or inhibit formation of dental caries, (ii) reduce, repair or inhibit pre-carious lesions of the enamel, e.g., as detected by quantitative light-induced fluorescence (QLF) or electrical caries measurement (ECM), (iii) reduce or inhibit demineralization and promote remineralization of the teeth, (iv) reduce hypersensitivity of the teeth, (v) reduce or inhibit gingivitis, (vi) promote healing of sores or cuts in the mouth, (vii) reduce levels of acid producing bacteria, (viii) to increase relative levels of arginolytic bacteria, (ix) inhibit microbial biofilm formation in the oral cavity, (x) raise and/or maintain plaque pH at levels of at least about pH 5.5 following sugar challenge, (xi) reduce plaque accumulation, (xii) treat, reduce or relieve dry mouth, (xiii) clean the teeth and oral cavity (xiv) reduce erosion, (xv) whiten teeth, (xvi) immunize the teeth against cariogenic bacteria, and/or (xvii) promote systemic health, including cardiovascular health, e.g., by reducing potential for systemic infection via the oral tissues comprising applying a Composition of the Invention to the oral cavity, e.g., by applying a Composition of the Invention to the oral cavity of a patient in need thereof.

The invention further comprises the use of a basic amino acid, e.g., arginine, in the manufacture of a Composition of the Invention, e.g., in accordance with any of the methods Method 2, or for use in any of the indications set forth in Method 3.

It may therefore be seen by the skilled practitioner in the oral care art that a surprising technical effect and advantage of forming a bicarbonate salt of a basic amino acid, such as arginine, in situ within the oral care composition, by reacting a bicarbonate precursor and the basic amino acid precursor in the composition itself, can be achieved, i.a. that a relatively expensive commercially available bicarbonate salt of a basic amino acid can be avoided without reducing the enhanced dental treatment of teeth provided by arginine.

### DETAILED DESCRIPTION OF THE INVENTION

Without being bound by theory, it is believed that oral care compositions comprising arginine bicarbonate, e.g., arginine and bicarbonate anions, may be formed by the addition of arginine free base and carbonate salts, e.g., sodium bicarbonate and sodium carbonate. The use of such materials proves to be a benefit from using arginine bicarbonate, as arginine free base and the carbonate salts are considerably cheaper to source than arginine bicarbonate.

The basic amino acids which can be used in the compositions and methods of the invention include not only naturally occurring basic amino acids, such as arginine, lysine, and histidine, but also any basic amino acids having a carboxyl group and an amino group in the molecule. Accordingly, basic amino acids include, but are not limited to, arginine, lysine, citrullene, ornithine, creatine, histidine, diaminobutanoic acid, diaminoproprionic acid, salts thereof or combinations thereof. In a particular embodiment, the basic amino acids are selected from arginine, citrullene, and ornithine. In certain embodiments, the basic amino acid is arginine, for example, 1-arginine, or a salt thereof.

In various embodiments, the basic amino acid is present in an amount of about 0.1 wt. % to about 20 wt. % of the total composition weight, about 1 wt. % to about 10 wt. % of the total composition weight, for example about 1.5 wt. %, about 3.75 wt. %, about 5 wt. %, or about 7.5 wt. % of the total composition weight.

The oral care compositions may further include one or more fluoride ion sources, e.g., fluoride salts which may be soluble. To enhance compatibility, fluoride salts wherein the fluoride is covalently bound to another atom and/or sequestered from calcium are preferred. A wide variety of fluoride ion-yielding materials can be employed as sources of soluble fluoride in the present compositions. Examples of suitable fluoride ion-yielding materials are found in U.S. Pat. No. 3,535,421, to Briner et al.; U.S. Pat. No. 4,885,155, to Parran, Jr. et al. and U.S. Pat. No. 3,678,154, to Widder et al., incorporated herein by reference.

Representative fluoride ion sources include, but are not limited to, stannous fluoride, sodium fluoride, potassium fluoride, sodium monofluorophosphate, sodium fluorosilicate, ammonium fluorosilicate, amine fluoride, ammonium fluoride, and combinations thereof. In certain embodiments the fluoride ion source includes stannous fluoride, sodium fluoride, sodium monofluorophosphate as well as mixtures thereof.

In certain embodiments, the oral care composition of the invention may also contain a source of fluoride ions or fluorine-providing ingredient in amounts sufficient to supply about 25 ppm to 25,000 ppm of fluoride ions, generally at least about 500 ppm, e.g., about 500 to about 2000 ppm, e.g., about 1000 to about 1600 ppm, e.g., about 1450 ppm. The appropriate level of fluoride will depend on the particular application. A mouthwash, for example, would typically have about 100 to about 250 ppm fluoride. A toothpaste for general consumer use would typically have about 1000 to about 1500 ppm, with pediatric toothpaste having somewhat less. A dentifrice or coating for professional application could have as much as 5,000 or even 25,000 ppm fluoride.

Fluoride ion sources may be added to the compositions of the invention at a level of about 0.01 wt. % to about 10 wt. % in one embodiment or about 0.03 wt. % to about 5 wt. %, and in another embodiment about 0.1 wt. % to about 1 wt. % by weight of the composition in another embodiment. Weights of fluoride salts to provide the appropriate level of fluoride ion will obviously vary based on the weight of the counter ion in the salt.

The Compositions of the Invention may comprise a calcium phosphate abrasive, e.g., tricalcium phosphate (Ca₃(PO₄)₂), hydroxyapatite (Ca₁₀(PO₄)₆(OH)₂), or dicalcium phosphate dihydrate (CaHPO₄.2H₂O, also sometimes referred to herein as DiCal) or calcium pyrophosphate.

The compositions may include one or more additional abrasives, for example silica abrasives such as precipitated silicas having a mean particle size of up to about 20 microns, such as Zeodent 115®, marketed by J. M. Huber. Other useful abrasives also include sodium metaphosphate, potassium metaphosphate, aluminum silicate, calcined alumina, bentonite or other siliceous materials, or combinations thereof.

The silica abrasive polishing materials useful herein, as well as the other abrasives, generally have an average particle size of about 0.1 and about 30 microns, about 5 and about 15 microns. The silica abrasives can be from precipitated silica or silica gels, such as the silica xerogels described in U.S. Pat. No. 3,538,230, to Pader et al. and U.S. Pat. No. 3,862,307, to Digidulio, both incorporated herein by reference. Particular silica xerogels are marketed under the trade name Syloid® by the W. R. Grace & Co., Davison Chemical Division. The precipitated silica materials include those marketed by the J. M. Huber Corp. under the name Zeodent®, including the silica carrying the designation Zeodent 115 and 119. These silica abrasives are described in U.S. Pat. No. 4,340,583, to Wason, incorporated herein by reference.

In certain embodiments, abrasive materials useful in the practice of the oral care compositions in accordance with the invention include silica gels and precipitated amorphous silica having an oil absorption value of about fess than 100 cc/100 g silica and in the range of about 45 cc/100 g to about 70 cc/100 g silica. Oil absorption values are measured using the ASTA Rub-Out Method D281. In certain embodiments, the silicas are colloidal particles having an average particle sire of about 3 microns to about 12 microns, and about 5 to about 10 microns.

In particular embodiments, the abrasive materials comprise a large fraction of very small particles, e.g., having a d50 less than about 5 microns, for example small particle silica (SPS) having a d50 of about 3 to about 4 microns, for example Sorbosil AC43® (Ineos). Such small particles are particularly useful in formulations targeted at reducing hypersensitivity. The small particle component may be present in combination with a second larger particle abrasive. In certain embodiments, for example, the formulation comprises about 3 about 8% SPS and about 25 to about 45% of a conventional abrasive.

Low oil absorption silica abrasives particularly useful in the practice of the invention are marketed under the trade designation Sylodent XWA® by Davison Chemical Division of W.R. Grace & Co., Baltimore, Md. 21203. Sylodent 650 XWA®, a silica hydrogel composed of particles of colloidal silica having a water content of about 29% by weight averaging about 7 to about 10 microns in diameter, and an oil absorption of less than about 70 cc/100 g of silica is an example of a low oil absorption silica abrasive useful in the practice of the present invention. The abrasive is present in the oral care composition of the present invention at a concentration of about 10 to about 60% by weight, in other embodiment about 20 to about 45% by weight, and in another embodiment about 30 to about 50% by weight.

The oral care compositions of the invention also may include an agent to increase the amount of foam that is produced when the oral cavity is brushed.

Illustrative examples of agents that increase the amount of foam include, but are not limited to polyoxyethylene and certain polymers including, but not limited to, alginate polymers.

The polyoxyethylene may increase the amount of foam and the thickness of the foam generated by the oral care carrier component of the present invention. Polyoxyethylene is also commonly known as polyethylene glycol (“PEG”) or polyethylene oxide. The polyoxyethylenes suitable for this invention will have a molecular weight of about 200,000 to about 7,000,000. In one embodiment the molecular weight will be about 600,000 to about 2,000,000 and in another embodiment about 800,000 to about 1,000,000. Polyox® is the trade name for the high molecular weight polyoxyethylene produced by Union Carbide.

The polyoxyethylene may be present in an amount of about 1% to about 90%, in one embodiment about 5% to about 50% and in another embodiment about 10% to about 20% by weight of the oral care carrier component of the oral care compositions of the present invention. The dosage of foaming agent in the oral care composition (i.e., a single dose) is about 0.01 to about 0.9% by weight, about 0.05 to about 0.5% by weight, and in another embodiment about 0.1 to about 0.2% by weight.

Another agent optionally included in the oral care composition of the invention is a surfactant or a mixture of compatible surfactants. Suitable surfactants are those which are reasonably stable throughout a wide pH range, for example, anionic, cationic, nonionic or zwitterionic surfactants.

Suitable surfactants are described more fully, for example, in U.S. Pat. No. 3,959,458, to Agricola et al.; U.S. Pat. No. 3,937,807, to Haefele; and U.S. Pat. No. 4,051,234, to Gieske et al., which are incorporated herein by reference.

In certain embodiments, the anionic surfactants useful herein include the water-soluble salts of alkyl sulfates having about 10 to about 18 carbon atoms in the alkyl radical and the water-soluble salts of sulfonated monoglycerides of fatty acids having about 10 to about 18 carbon atoms. Sodium lauryl sulfate, sodium lauroyl sarcosinate and sodium coconut monoglyceride sulfonates are examples of anionic surfactants of this type. Mixtures of anionic surfactants may also be utilized.

In another embodiment, cationic surfactants useful in the present invention can be broadly defined as derivatives of aliphatic quaternary ammonium compounds having one long alkyl chain containing about 8 to about 18 carbon atoms such as lauryl trimethylammonium chloride, cetyl pyridinium chloride, cetyl trimethylammonium bromide, di-isobutylphenoxyethyldimethylbenzylammonium chloride, coconut alkyltrimethylammonium nitrite, cetyl pyridinium fluoride, and mixtures thereof.

Illustrative cationic surfactants are the quaternary ammonium fluorides described in U.S. Pat. No. 3,535,421, to Briner et al., herein incorporated by reference. Certain cationic surfactants can also act as germicides in the compositions.

Illustrative nonionic surfactants that can be used in the compositions of the invention can be broadly defined as compounds produced by the condensation of alkylene oxide groups (hydrophilic in nature) with an organic hydrophobic compound which may be aliphatic or alkylaromatic in nature. Examples of suitable nonionic surfactants include, but are not limited to, the Pluronics, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and mixtures of such materials.

In certain embodiments, zwitterionic synthetic surfactants useful in the present invention can be broadly described as derivatives of aliphatic quaternary ammonium, phosphomium, and sulfonium compounds, in which the aliphatic radicals can be straight chain or branched, and wherein one of the aliphatic substituents contains about 8 to about 18 carbon atoms and one contains an anionic water-solubilizing group, e.g., carboxy, sulfonate, sulfate, phosphate or phosphonate. Illustrative examples of the surfactants suited for inclusion into the composition include, but are not limited to, sodium alkyl sulfate, sodium lauroyl sarcosinate, cocoamidopropyl betaine and polysorbate 20, and combinations thereof.

In a particular embodiment, the Composition of the Invention comprises an anionic surfactant, e.g., sodium lauryl sulfate.

The surfactant or mixtures of compatible surfactants can be present in the compositions of the present invention in about 0.1% to about 5.0%, in another embodiment about 0.3% to about 3.0% and in another embodiment about 0.5% to about 2.0% by weight of the total composition.

The oral care compositions of the invention may also include a flavoring agent. Flavoring agents which are used in the practice of the present invention include, but are not limited to, essential oils as well as various flavoring aldehydes, esters, alcohols, and similar materials. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Certain embodiments employ the oils of peppermint and spearmint.

The flavoring agent is incorporated in the oral composition at a concentration of about 0.1 to about 5% by weight and about 0.5 to about 1.5% by weight. The dosage of flavoring agent in the individual oral care composition dosage (i.e., a single dose) is about 0.001 to about 0.05% by weight and in another embodiment about 0.005 to about 0.015% weight.

The oral care compositions of the invention also may optionally include one or more chelating agents able to complex calcium found in the cell walls of the bacteria. Binding of this calcium weakens the bacterial cell wall and augments bacterial lysis.

Another group of agents suitable for use as chelating agents in the present invention are the soluble pyrophosphates. The pyrophosphate salts used in the present compositions can be any of the alkali metal pyrophosphate salts. In certain embodiments, salts include tetra alkali metal pyrophosphate, dialkali metal diacid pyrophosphate, trialkali metal monoacid pyrophosphate and mixtures thereof, wherein the alkali metals are sodium or potassium. The salts are useful in both their hydrated and unhydrated forms. An effective amount of pyrophosphate salt useful in the present composition is generally enough to provide at least about 1 wt. % pyrophosphate ions, about 1.5 wt. % to about 6 wt. %, about 3.5 wt. % to about 6 wt. % of such ions.

The oral care compositions of the invention also optionally include one or more polymers, such as polyethylene glycols, polyvinylmethyl ether maleic acid copolymers, polysaccharides (e.g., cellulose derivatives, for example carboxymethyl cellulose, or polysaccharide gums, for example xanthan gum or carrageenan gum). Acidic polymers, for example polyacrylate gels, may be provided in the form of their free acids or partially or fully neutralized water soluble alkali metal (e.g., potassium and sodium) or ammonium salts. Certain embodiments include about 1:4 to about 4:1 copolymers of maleic anhydride or acid with another polymerizable ethylenically unsaturated monomer, for example, methyl vinyl ether (methoxyethylene) having a molecular weight (M.W.) of about 30,000 to about 1,000,000. These copolymers are available for example as Gantrez AN 139 (M.W. 500,000), AN 119 (M.W. 250,000) and S-97 Pharmaceutical Grade (M.W. 70,000), of GAF Chemicals Corporation.

Other operative polymers include those such as the 1:1 copolymers of maleic anhydride with ethyl acrylate, hydroxyethyl methacrylate, N-vinyl-2-pyrollidone, or ethylene, the latter being available for example as Monsanto EMA No. 1103, M.W. 10,000 and EMA Grade 61, and 1:1 copolymers of acrylic acid with methyl or hydroxyethyl methacrylate, methyl or ethyl acrylate, isobutyl vinyl ether or N-vinyl-2-pyrrolidone.

Suitable generally, are polymerized olefinically or ethylenically unsaturated carboxylic acids containing an activated carbon-to-carbon olefinic double bond and at least one carboxyl group, that is, an acid containing an olefinic double bond which readily functions in polymerization because of its presence in the monomer molecule either in the alpha-beta position with respect to a carboxyl group or as part of a terminal methylene grouping. Illustrative of such acids are acrylic, methacrylic, ethacrylic, alpha-chloroacrylic, crotonic, beta-acryloxy propionic, sorbic, alpha-chlorsorbic, cinnamic, beta-styrylacrylic, muconic, itaconic, citraconic, mesaconic, glutaconic, aconitic, alpha-phenylacrylic, 2-benzyl acrylic, 2-cyclohexylacrylic, angelic, umbellic, fumaric, maleic acids and anhydrides. Other different olefinic monomers copolymerizable with such carboxylic monomers include vinylacetate, vinyl chloride, dimethyl maleate and the like. Copolymers contain sufficient carboxylic salt groups for water-solubility.

A further class of polymeric agents includes a composition containing homopolymers of substituted acrylamides and/or homopolymers of unsaturated sulfonic acids and salts thereof, in particular where polymers are based on unsaturated sulfonic acids selected from acrylamidoalykane sulfonic acids such as 2-acrylamide 2 methylpropane sulfonic acid having a molecular weight of about 1,000 to about 2,000,000, described in U.S. Pat. No. 4,842,847, Jun. 27, 1989 to Zahid, incorporated herein by reference.

Another useful class of polymeric agents includes polyamino acids, particularly those containing proportions of anionic surface-active amino acids such as aspartic acid, glutamic acid and phosphoserine, as disclosed in U.S. Pat. No. 4,866,161 Sikes et al., incorporated herein by reference.

In preparing oral care compositions, it is sometimes necessary to add some thickening material to provide a desirable consistency or to stabilize or enhance the performance of the formulation. In certain embodiments, the thickening agents are carboxyvinyl polymers, carrageenan, hydroxyethyl cellulose and water soluble salts of cellulose ethers such as sodium carboxymethyl cellulose and sodium carboxymethyl hydroxyethyl cellulose. Natural gums such as karaya, gum arabic, and gum tragacanth can also be incorporated. Colloidal magnesium aluminum silicate or finely divided silica can be used as component of the thickening composition to further improve the composition's texture. In certain embodiments, thickening agents in an amount of about 0.5% to about 5.0% by weight of the total composition are used.

The oral care compositions of the invention may also optionally include one or more enzymes. Useful enzymes include any of the available proteases, glucanohydrolases, endoglycosidases, amylases, mutanases, lipases and mucinases or compatible mixtures thereof. In certain embodiments, the enzyme is a protease, dextranase, endoglycosidase and mutanase. In another embodiment, the enzyme is papain, endoglycosidase or a mixture of dextranase and mutanase. Additional enzymes suitable for use in the present invention are disclosed in U.S. Pat. No. 5,000,939 to Dring et al., U.S. Pat. No, 4,992,420; U.S. Pat. No. 4,355,022; U.S. Pat. No. 4,154,815; U.S. Pat. No. 4,058,595; U.S. Pat. No, 3,991,177; and U.S. Pat. No. 3,696,191 all incorporated herein by reference. An enzyme of a mixture of several compatible enzymes in the current invention constitutes about 0.002% to about 2% in one embodiment or about 0.05% to about 1.5% in another embodiment or in yet another embodiment about 0.1% to about 0.5%.

Water may also be present in the oral compositions of the invention. Water, employed in the preparation of commercial oral compositions should be deionized and free of organic impurities. Water commonly makes up the balance of the compositions and includes about 10% to about 90%, about 20% to about 60% or about 10% to about 30% by weight of the oral compositions. This amount of water includes the free water which is added plus that amount which is introduced with other materials such as with sorbitol or any components of the invention.

Within certain embodiments of the oral compositions, it is also desirable to incorporate a humectant to prevent the composition from hardening upon exposure to air. Certain humectants can also impart desirable sweetness or flavor to dentifrice compositions. The humectant, on a pure humectant basis, generally includes about 15% to about 70% in one embodiment or about 30% to about 65% in another embodiment by weight of the dentifrice composition.

Suitable humectants include edible polyhydric alcohols such as glycerine, sorbitol, xylitol, propylene glycol as well as other polyols and mixtures of these humectants. Mixtures of glycerine and sorbitol may be used in certain embodiments as the humectant component of the toothpaste compositions herein.

In addition to the above described components, the embodiments of this invention can contain a variety of optional dentifrice ingredients some of which are described below. Optional ingredients include, for example, but are not limited to, adhesives, sudsing agents, flavoring agents, sweetening agents, additional antiplaque agents, abrasives, and coloring agents. These and other optional components are further described in U.S. Pat. No. 5,004,597, to Majeti; U.S. Pat. No. 3,959,458 to Agricola et al. and U.S. Pat. No. 3,937,807, to Haefele, all being incorporated herein by reference.

The compositions of the present invention can be made using methods which are common in the oral product area.

The present invention in its method aspect involves applying to the oral cavity a safe and effective amount of the compositions described herein.

The compositions and methods according to the invention are useful to a method to protect the teeth by facilitating repair and remineralization, in particular to reduce or inhibit formation of dental caries, reduce or inhibit demineralization and promote remineralization of the teeth, reduce hypersensitivity of the teeth, and reduce, repair or inhibit pre-carious lesions of the enamel, e.g., as detected by quantitative light-induced fluorescence (QLF) or electrical caries measurement (ECM). Quantitative light-induced fluorescence is a visible light system that permits early detection of pre-carius lesions in the enamel. Normal teeth fluoresce in visible light; demineralized teeth do not or do so only to a lesser degree. The area of demineralization can be quantified and its progress monitored. Electrical conductance measurement exploits the fact that the fluid-tilled tubules exposed upon demineralization and erosion of the enamel conduct electricity. An increase in the conductance of the patient's teeth therefore may indicate demineralization. The Compositions of the Invention are thus useful in a method to reduce pre-carious lesions of the enamel (as measured by QLF or ECM) relative to a composition lacking effective amounts of fluorine and/or arginine.

The Compositions of the invention are additionally useful in methods to reduce harmful bacteria in the oral cavity, for example methods to reduce or inhibit gingivitis, reduce levels of acid producing bacteria, to increase relative levels of arginolytic bacteria, inhibit microbial biofilm formation in the oral cavity, raise and/or maintain plaque pH at levels of about at least pH 5.5, reduce plaque accumulation, and/or clean the teeth and oral cavity.

Finally, by increasing the pH in the mouth and discouraging pathogenic bacteria, the Compositions of the Invention are useful to promote healing of sores or cuts in the mouth.

The compositions and methods according to the invention can be incorporated into oral compositions for the care of the mouth and teeth such as toothpastes, transparent pastes, gels, mouth rinses, sprays and chewing gum.

Levels of active ingredients will vary based on the nature of the delivery system and the particular active. For example, the basic amino acid may be present at levels from, e.g., about 0.1 to about 20 wt % (expressed as weight of free base), e.g., about 0.1 to about 3 wt % for a mouthrinse, about 1 to about 10 wt % for a consumer toothpaste or about 7 to about 20 wt % for a professional or prescription treatment product. Fluoride may be present at levels of, e.g., about 25 to about 25,000 ppm, for example about 25 to about 250 ppm for a mouthrinse, about 750 to about 2,000 ppm for a consumer toothpaste, or about 2,000 to about 25,000 ppm. for a professional or prescription treatment product. Levels of antibacterial will vary similarly, with levels used in toothpaste being e.g., about 5 to about 15 times greater than used in mouthrinse. For example, a triclosan mouthrinse may contain, e.g., about 0.03 wt % triclosan while a triclosan toothpaste may contain about 0.3 wt % triclosan.

Enhancing oral health also provides benefits in systemic health, as the oral tissues can be gateways for systemic infections. Good oral health is associated with systemic health, including cardiovascular health. The compositions and methods of the invention provide particular benefits because basic amino acids, especially arginine, are sources of nitrogen which supply NO synthesis pathways and thus enhance microcirculation in the oral tissues. Providing a less acidic oral environment is also helpful in reducing gastric distress and creates an environment less favorable to Heliobacter, which is associated with gastric ulcers. Arginine in particular is required for high expression of specific immune cell receptors, for example T-cell receptors, so that arginine can enhance an effective immune response. The compositions and methods of the invention are thus useful to enhance systemic health, including cardiovascular health.

As used throughout, ranges are used as shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls. It is understood that when formulations are described, they may be described in terms of their ingredients, as is common in the art, notwithstanding that these ingredients may react with one another in the actual formulation as it is made, stored and used, and such products are intended to be covered by the formulations described.

The following examples further describe and demonstrate illustrative embodiments within the scope of the present invention. The examples are given solely for illustration and are not to be construed as limitations of this invention as many variations are possible without departing from the spirit and scope thereof. Various modifications of the invention in addition to those shown and described herein should be apparent to those skilled in the art and are intended to fall within the appended claims.

### Example 1

A premix consisting of 4.26 g heavy water (D-₂O), 0.40 g arginine and 0.24 g sodium bicarbonate is prepared, having an initial pH of 9.74. The premix is adjusted to a pH of 8.99 with a 34% HCl solution. Proton NMR is used to record the spectra, and show arginine bicarbonate complex.

### Example 2

A premix consisting of 4.26 D₂O, 0.40 g L-arginine and 0.31 sodium carbonate is prepared, having an initial pH of 11.94. The premix is adjusted to a pH of 9.01 with a 34% HCl solution. Proton NMR is used to record the spectra, and show an arginine bicarbonate complex.

1. An oral care composition comprising a basic amino acid in free or salt form and a soluble carbonate salt, wherein a bicarbonate of the basic amino acid is formed in situ.

2. The oral care composition of claim 1 having a pH of from about 8.5 to about 9.5.

3. The oral care composition of claim 1, wherein the basic amino acid is arginine, lysine, citrullene, ornithine, creatine, histidine, diaminobutanoic acid, diaminoproprionic acid, salts thereof and/or combinations thereof.

4. The oral care composition of claim 3, wherein the basic amino acid is arginine.

5. The oral care composition of claim 1, wherein the basic amino acid is present in an amount corresponding to about 1 wt. % to about 10 wt. % of the total composition weight.

6. The oral care composition of claim 1, wherein the soluble carbonate salt is selected from sodium carbonate, sodium bicarbonate, and mixtures thereof.

7. The oral care composition of claim 1, further comprising an effective amount of a fluoride ion source.

8. The oral care composition claim 1, further comprising an abrasive.

9. The oral care composition of claim 8 wherein the abrasive is selected from precipitated calcium carbonate, silica and mixtures thereof.

10. The oral care composition of claim 1, further comprising at least one surfactant.

11. The oral care composition of claim 1, further comprising at least one humectant.

12. The oral care composition of claim 1, further comprising an antibacterial agent.

13. The oral care composition of claim 1, further comprising a physiologically acceptable potassium salt in an amount effective to reduce dentinal sensitivity.

14. The oral care composition of claim 1, wherein the composition is toothpaste.

15. The oral care composition of claim 1, wherein the composition is a mouthwash.

16. A method for preparing an oral care composition comprising a bicarbonate salt of a basic amino acid comprising mixing a basic amino acid in free or salt form and a soluble carbonate salt.

17. The method of claim 16 wherein the mixture comprises about 7 to about 10% wt, of the basic amino acid, weight being given as a free base.

18. The method of claim 16 wherein the carbonate salt is selected from sodium carbonate, sodium bicarbonate and mixtures thereof.

19. The method of claim 16 wherein the molar ratio of arginine to bicarbonate ion is about 4:1 to about 1:4.

20. The method of claim 16 wherein the composition is adjusted to about pH 9.

21. The method of claim 16 wherein the basic amino acid is selected from arginine, lysine, citrullene, ornithine, creatine, histidine, diaminobutanoic acid, diaminoproprionic acid, salts thereof and/or combinations thereof.

22. The method of claim 21 wherein the basic amino acid is arginine in free or salt form or mixtures thereof.

23. The method of claim 22 wherein the arginine is initially provided in hydrochloride salt form.

24. The method of claim 16 further comprising adjusting the pH to about 8.5 to about 9.5.

25. The method of claim 24 wherein the oral care composition further comprises secondary materials are selected from the group consisting of a fluoride ion source, abrasives, surfactants, humectants, antibacterial agents, calcium salts, potassium salts, and combinations thereof.

26. An oral care composition prepared by the method of claim 16.

27. A method comprising applying an effective amount of the oral care composition of claim 1 to the oral cavity of a subject in need thereof to:
(i) reduce or inhibit formation of dental caries,
(ii) reduce, repair or inhibit pre-carious lesions of the enamel,
(iii) reduce or inhibit demineralization and promote remineralization of the teeth,
(iv) reduce hypersensitivity of the teeth,
(v) reduce or inhibit gingivitis,
(vi) promote healing of sores or cuts in the mouth,
(vii) reduce levels of acid producing bacteria,
(viii) increase relative levels of arginolytic bacteria,
(ix) inhibit microbial biofilm formation in the oral cavity,
(x) raise and/or maintain plaque pH at levels of at least pH 5.5 following sugar challenge,
(xi) reduce plaque accumulation,
(xii) treat, relieve or reduce dry mouth,
(xiii) clean the teeth and oral cavity
(xiv) reduce erosion,
(xv) whiten teeth,
(xvi) immunize the teeth against cariogenic bacteria; and/or
(xvii) promote systemic health, including cardiovascular health.

28. The oral care composition of claim 7, wherein the fluoride ion source is selected from stannous fluoride, sodium fluoride, potassium fluoride, sodium monofluorophosphate, sodium fluorosilicate, ammonium fluorosilicate, amine fluoride, ammonium fluoride, and combination of two or more thereof.

29. The oral care composition of claim 13, wherein said potassium salt is selected from potassium nitrate and potassium chloride.

30. The method of claim 25, wherein at least one of said secondary materials is a fluoride ion source selected from stannous fluoride, sodium fluoride, potassium fluoride, sodium monofluorophosphate, sodium fluorosilicate, ammonium fluorosilicate, amine fluoride, ammonium fluoride, and combination of two or more thereof.

31. The method of claim 25, wherein at least one of said secondary materials is a potassium salt selected from potassium nitrate and potassium chloride.
